(19) United States
(12) Patent Application Publication
(10) Pub. No.: US 2008/0281224 A1
(43) Pub. Date: Nov. 13, 2008

(54) BIOPSY DEVICE NEEDLE TIP

(76) Inventor: Michael E. JOHNSON, West Chester, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/117,933

(22) Filed: May 9, 2008

Related U.S. Application Data
(60) Provisional application No. 60/917,375, filed on May 11, 2007.

(51) Int. Cl. A61B 10/02

(52) U.S. Cl. 600/567

(57) ABSTRACT

A biopsy device having a cannula with a distal tip is disclosed. The distal tip includes a blade, and can be a unitary metal injection molded component including a base and a blade. The blade has a hardness of at least 40 HRC. The blade can be hardened, polished, and then honed to provide a sharp leading edge.

### REFERENCE TO RELATED APPLICATIONS

This application claims the benefit of U.S. Provisional Application Ser. No. 60/917,375 entitled “BIOPSY DEVICE NEEDLE TIP”, Filed on May 11, 2007. This application cross references and incorporates by reference U.S. patent application Ser. No. 10/785,755, filed Feb. 24, 2004, entitled “Biopsy Device with Variable Speed Cutter Advance,” issued as U.S. Pat. No. 7,025,732.

This application cross references and incorporates by reference “Method for Biopsy Device Needle Tip” filed on even date herewith in the name of Michael E. Johnson.

### FIELD OF THE INVENTION

The present invention relates in general to biopsy devices and, and more particularly to tips, such as bladed tips, used with biopsy devices, and methods of making such tips.

### BACKGROUND OF THE INVENTION

The diagnosis and treatment of patients with cancerous tumors is an ongoing area of investigation. Medical devices for obtaining tissue samples for subsequent sampling are known in the art. For instance, a biopsy instrument now marketed under the tradename MAMMOTOME is commercially available for use in obtaining breast biopsy samples.

The following patent documents disclose various biopsy devices and are incorporated herein by reference in their entirety: U.S. Pat. No. 7,025,732 issued Apr. 11, 2006; U.S. Pat. No. 6,273,862 issued Aug. 14, 2001; U.S. Pat. No. 6,231,522 issued May 15, 2001; U.S. Pat. No. 6,228,055 issued May 8, 2001; U.S. Pat. No. 6,120,462 issued Sep. 19, 2000; U.S. Pat. No. 6,086,544 issued Jul. 11, 2000; U.S. Pat. No. 6,077,230 issued Jun. 20, 2000; U.S. Pat. No. 6,017,316 issued Jan. 25, 2000; U.S. Pat. No. 6,007,497 issued Dec. 28, 1999; U.S. Pat. No. 5,980,469 issued Nov. 9, 1999; U.S. Pat. No. 5,964,716 issued Oct. 12, 1999; U.S. Pat. No. 5,928,164 issued Jul. 27, 1999; U.S. Pat. No. 5,775,333 issued Jul. 7, 1998; U.S. Pat. No. 5,769,086 issued Jun. 23, 1998; U.S. Pat. No. 5,649,547 issued Jul. 22, 1997; U.S. Pat. No. 5,526,822 issued Jun. 18, 1996; US 2003/0199785 published Oct. 23, 2003; US 2003/0199754 published Oct. 23, 2003; US 2003/0199754 published Oct. 23, 2003.

Biopsy devices may include a cannula having a distal tip, such as a distal tip including a blade. The blade of the distal tip is typically used to assist in piercing skin and/or advancing the cannula into a tissue mass, so that a tissue sample may be obtained with the biopsy device. The biopsy device may be a mounted device, such as for stereotactic applications, and include a firing mechanism for directing the distal tip into tissue. Alternatively, the biopsy device may be a handheld device, such as for use with ultrasound, in which case the physician directs the tip of the device into tissue and manipulates the device with a single hand. In either case, it is desirable to reduce the force required to penetrate tissue.

### SUMMARY OF THE INVENTION

In one embodiment, the invention provides a biopsy device having a distal tip including a metallic blade having a hardness of at least 40 RHC (Rockwell Hardness C scale), and more particularly, between about 43 HRC to about 45 HRC. The distal tip can be a metal injection molded (MIM) component that includes a base and an integral flat blade.

The blade can have a sharp leading edge that is formed by grinding or honing, and the leading edge can have a leading edge included angle of less than about 45 degrees, and more particularly, less than or equal to about 42 degrees.

The blade can include a ground or honed surface extending from the leading edge, with a plurality of serrations extending on the surface from the leading edge, the serrations being generally aligned with the longitudinal axis of the cannula. In one embodiment, the blade has honed surfaces on oppositely facing sides of the blade, with each honed surface including a plurality of serrations generally aligned with the longitudinal axis of the cannula.

A method is provided for forming a blade for a biopsy device. The method can include the steps of providing a distal tip comprising a generally flat blade portion extending from a base portion adapted for attachment to the cannula; hardening the blade portion (such as by heat treating the distal tip) to have a hardness of at least about 40 HRC; and forming a sharp blade leading edge, such as by honing or grinding.

The step of hardening can include heat treating the blade portion. The method can include the step of honing the blade to form a sharp leading edge after the step of hardening the blade. The method can also include polishing the blade, such as by electropolishing, after hardening the blade and prior to honing the blade. In one embodiment, the distal tip is heat treated, and after heat treating, the distal tip is attached to the cannula of the biopsy device. The distal tip and cannula can be polished by electro polishing. After electropolishing, the distal tip mounted on the cannula can be advanced toward a honing apparatus in a direction generally parallel to the longitudinal axis of the cannula. Portions of the two sides of the blade can be honed simultaneously to form a sharp leading edge on the blade.

### BRIEF DESCRIPTION OF THE DRAWINGS

While the specification concludes with claims particularly pointing out and distinctly claiming the present invention, it is believed the same will be better understood by reference to the following description, taken in conjunction with the accompanying drawings in which:

FIG. 1 is an isometric view of a handheld biopsy device having a cannula, a distal tip with a generally flat blade attached to the distal end of the cannula, and an inner cutter advancable and rotatable within the cannula for severing tissue.

FIG. 2 is a schematic illustration of a prior art distal tip which can be used with the biopsy device of FIG. 1.

FIG. 3 is an isometric view of the distal end of a cannula having a distal tip according to the present invention.

FIG. 3A is a side view of the distal tip of FIG. 3.

FIG. 3B is a top view of the distal tip of FIG. 3 and illustrating the blade having a sharp leading edge formed to have an included angle IA.

FIG. 4 is an isometric view of a metal injection molded (MIM) distal tip component, which according to one aspect of the present invention can be hardened, such as by heat treatment, prior to forming a sharp leading edge on the blade portion.

FIG. 5 is an isometric view of the distal tip component of FIG. 4 attached to the distal end of the cannula.

FIG. 6 illustrates advancing the assembly shown in FIG. 5 relative to a honing apparatus to provide a sharp leading edge on the blade.

### DETAILED DESCRIPTION OF THE INVENTION

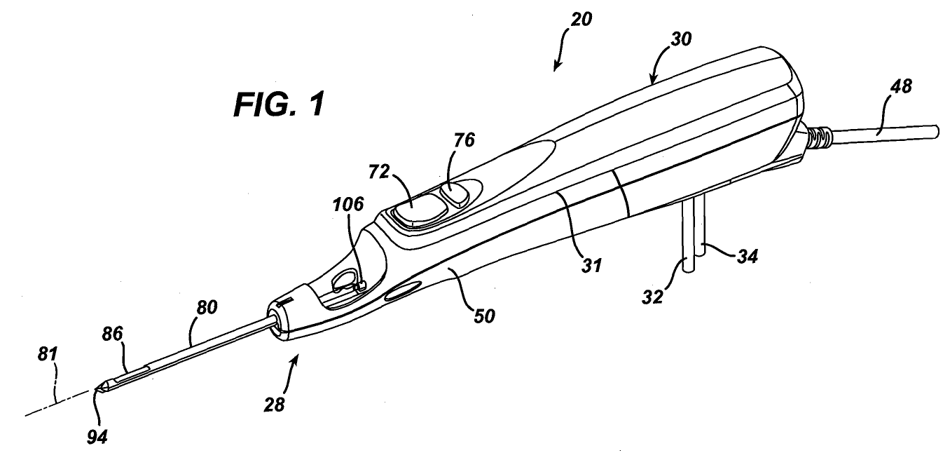

FIG. 1 illustrates a biopsy device comprising a handpiece identified generally by the numeral 20. One such biopsy device is a Mammotome® brand biopsy device marketed by Ethicon Endo-Surgery, Inc. Handpiece 20 is preferably lightweight and ergonomically-shaped to be easily manipulated by an operator's hand.

Handpiece 20 can include a disposable probe assembly 28 and a detachably connected holster 30. Probe assembly 28 can be detachable from holster 30 along interface 31. Probe assembly 28 can be connected to a vacuum system, such as by first vacuum tube 32 and second vacuum tube 34. Holster 30 can include a control cord 48 operationally connecting the handpiece 20 to a control unit and power source. While FIG. 1 illustrates a handheld biopsy device, mounted biopsy devices, such as those adapted to be mounted to a table for use with stereotactic systems can also be employed in connection with the description set forth below.

Holster 30 can include one or more switches to enable the operator to use the handpiece 20 with a single hand. These switches can include a rocker switch 72 for actuating the motion of a cutter (such as hollow tubular cutter) and a vacuum switch 76 for actuating a vacuum system. One-handed operation allows the operator's other hand to be free, for example, to hold an ultrasonic imaging device, or to deploy a biopsy marker to mark a biopsy site.

Probe assembly 28 can include a body including an outer shell 50. Outer shell 50 can be formed of one or more segments which may be injection molded from a rigid, biocompatible plastic, such as a polycarbonate. The outer shell 50 can be shaped to define a recess 73 for retrieving tissue samples extracted by probe assembly 28.

Probe assembly 28 can include a hollow outer cannula 80 extending distally from the outer shell 50. The hollow cannula 80 can have a distal tip 94 for piercing tissue and/or aiding in advancement of the hollow cannula 80 into a patient's breast tissue to obtain a tissue sample. Cannula 80 includes one or more internal lumen, such as an internal cutter lumen, and a lateral tissue receiving port 86 communicating with the internal cutter lumen of the cannula 80. The cannula 80 can extend along a longitudinal axis 81, shown schematically in FIG. 1. An inner cutter adapted for translation and rotation within the cutter lumen of cannula 80 is employed for severing tissue samples from a tissue mass received in tissue receiving port 86. In FIG. 1, the distal end 106 of the cutter is shown in a retracted position, proximal of the cannula 80. Operation of the internal cutter is described in U.S. Pat. No. 7,025,732, incorporated herein by reference.

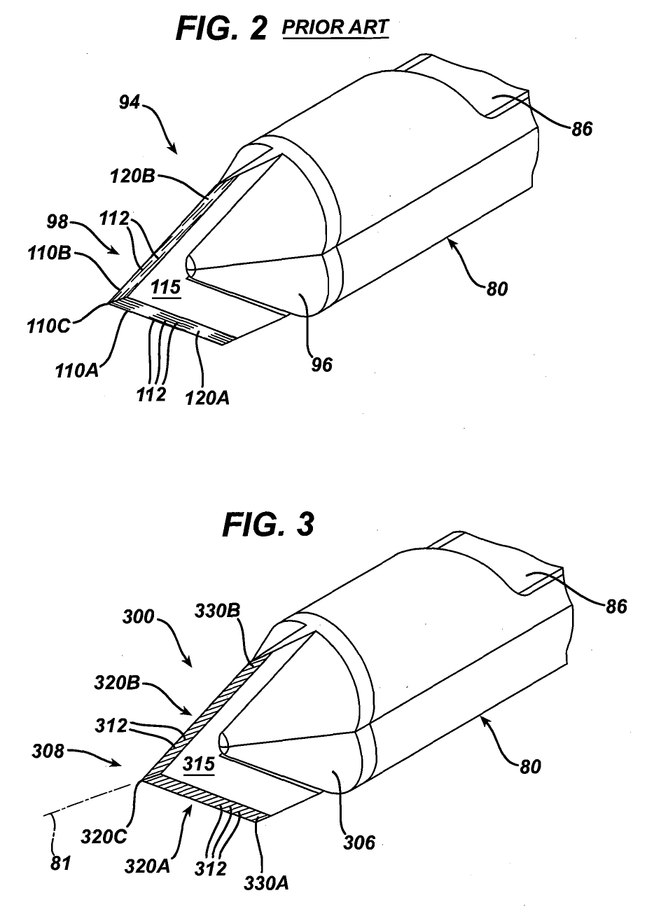

FIG. 2 illustrates a prior art distal tip 94 which can be used with the biopsy device shown in FIG. 1. Distal tip 94 can be a metal injection molded (MIM) component which is attached, such as by welding, gluing, brazing, or other suitable joining methods to the distal end of outer cannula 80.

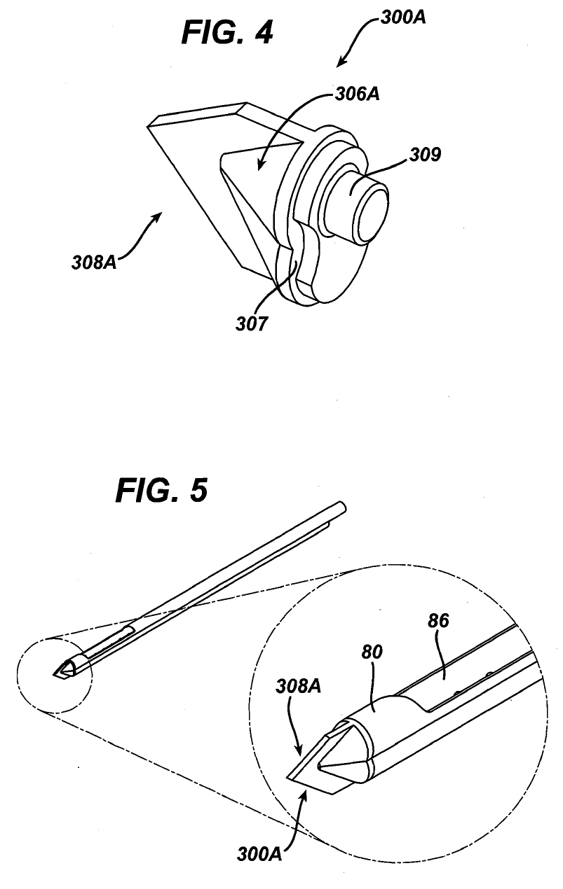

The distal tip 94 can be formed from a metal injection molded component, molded to have the shape generally similar to that shown in FIG. 4. Distal tip 94 includes a generally conical base 96 and a generally flat blade 98 extending distally from the base 96. The blade 98 includes a sharpened leading edge which includes upper leading edge portion 110B and lower leading edge portion 110A. The leading edge portions 110A and 110B meet at edge tip 110C, and are formed at the edge of ground facet surfaces 120A and 120B. The edge portions 110A and 110B extend proximally and radially from tip 110C, and lie in an imaginary plane that includes axis 81 of cannula 80. The upper facet surface 120B and lower facet surface 120A are ground in side walls 115 on each side of the blade 98 (only one side shown in FIG. 2). In the case of prior art distal tip 94 shown in FIG. 2, the facet surfaces 120A and 120B are ground in the side walls 115 prior to joining the distal tip 94 to the cannula. The facet surfaces 120A and 120B are ground in a direction generally parallel to the leading edge portions 110A and 110B, respectively, resulting in any grinding ridges 112 which extend generally parallel to the leading edge portions 110A and 110B, as shown in FIG. 2. As a result, the ridges 112 or other features formed by grinding facet surface 120A and 120B do not generally extend from leading edges 110A and 110B Further, in the prior art tip 94 shown in FIG. 2, the leading edge is formed in the blade portion of the tip 94 without hardening of the metal injection molded tip 94.

While the distal tip 94 shown in FIG. 2 is effective for use with the biopsy device in FIG. 1, Applicants have found that a surprisingly improved piercing blade can be provided as set forth below. In particular, the distal tip described below provides a reduced force (either hand held force or force applied by firing mechanism) to penetrate soft tissue, such as breast tissue.

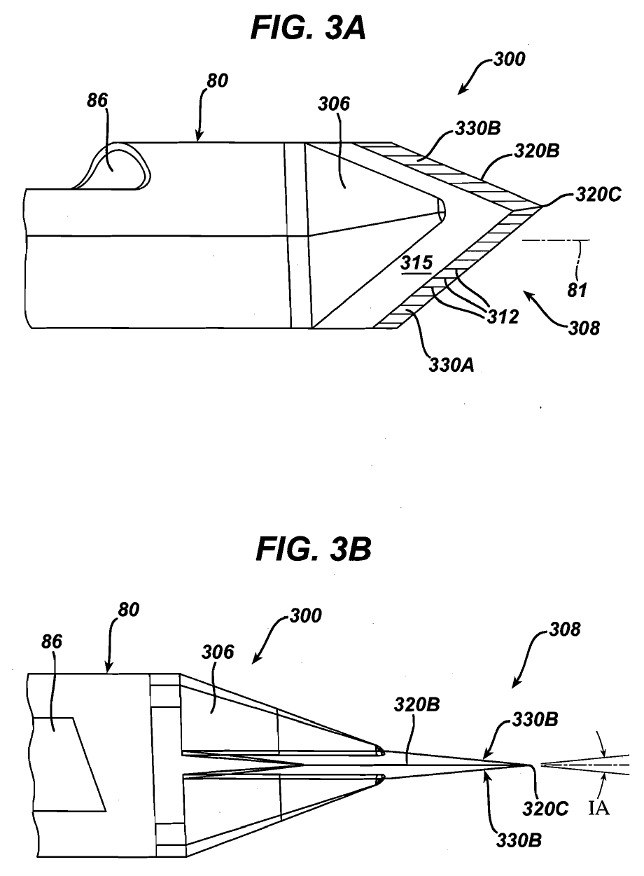

Referring to FIG. 3, an improved distal tip 300 is shown attached to the distal end of cannula 80. Distal tip 300 can include a conical base 306 and a generally flat blade 308 extending distally from base 306. The distal tip 300 can be hardened, such as by heat treating, so that the blade has a hardness of at least about 40 HRC (Hardness as measured on Rockwell Scale C). In one embodiment, the blade can be hardened by heat treatment to have a hardness of between about 43 HRC and about 45 HRC.

The blade 308 has side walls 315, and includes a sharpened leading edge which includes upper leading edge portion 320B and lower leading edge portion 320A. The leading edge portions 320A and 320B meet at edge tip 320C, and extend proximally and radially from tip 320C to lie in an imaginary plane that contains the longitudinal axis 81 of the cannula.

The sharp leading edge portions 320A and 320B are disposed along the edges of facet surfaces 330A and 330B. Facet surfaces 330A and 330 B are formed in side walls 315 on each side of the blade. For instance, the facet surfaces 330A and 330B and sharp leading edge portions 320A and 320B can be formed by grinding and/or honing the perimeter of the side walls 315 on each side of the blade 308. In one embodiment, the lower facet surfaces 330A can be honed on each side of the blade simultaneously. Likewise the upper facet surfaces 330B can be honed on each side of the blade simultaneously.

The side walls 315 can be honed to create facet surfaces 330A and 330B having serrations 312. As shown in FIG. 3 and FIG. 3A, the serrations 312 are generally aligned with the longitudinal axis 81 of the cannula 80. By “generally aligned with the longitudinal axis 81” it is meant that, when viewed from the side as shown in FIG. 3A, the serrations 312 extend generally parallel to the longitudinal axis 81 of the cannula 80 (Stated differently, if the serrations 312 are projected onto an imaginary plane containing sharp leading edge portions 320A and 320B, the projections of the serrations are substantially parallel to the longitudinal axis 81 of the cannula 80). Without being limited by theory, it is believed that having serrations (such as formed by grinding or honing) aligned with the longitudinal axis 81 (and direction of penetration) of the cannula 80 aids in reducing the force to penetrate skin and/or a tissue mass.

Further, as shown in FIG. 3A, a plurality of serrations 312 extend from each of the sharp leading edges 320A, 320B. Both sides of blade 308 can be honed to provide serrations 312 (and facets 330A and 330B) on both sides of blade 308. The intersection of the serrations 312 with the leading edges 320A,B provide a microscopically jagged, leading edge 320 having a plurality of microscopic “teeth” which correspond to the intersection of the serrations 312 with the leading edge 320. Such a leading edge can assist in cutting/penetrating tissue. The teeth so formed can be viewed under suitable magnification, or measured using a suitable stylus measurement device and/or atomic force microscope. In an alternative embodiment, serrations can be formed to extend from the leading edge by micro machining or electro discharge machining (EDM).

If desired, the geometry of the sharp leading edge 320 and facet surfaces (for instance the teeth and serrations 312) can be measured or quantified by any suitable means for measuring edge or surface topography, such as with an atomic force microscope having a probe with a cone angle of 20 degrees, a tip height of 15-20 micron, and a tip curvature radius of 10 nanometer.

Referring to FIG. 3B, the blade 308 can be honed to have a sharp leading edge with an included angle IA (as illustrated in FIG. 3B) of less than about 60 degrees, more particularly less than about 50 degrees, and still more particularly less than about 45 degrees.

The included angle IA is measured with respect to the facet surfaces on opposite sides of the blade. For example, in FIG. 3B the included angle IA provides a measure of the orientation of facet surfaces 330B on opposite sides of the blade. The included angle IA can be measured under suitable magnification. In one embodiment, the angle IA can be less than or equal to about 42 degrees. Without being limited by theory, it is believed that serrations 312 as shown in FIG. 3A and a relatively small included angle IA provide reduction in the force to penetrate tissue.

The blade 308 can be formed integrally with the base 306, such as by metal injection molding of the distal tip 300. A metal injection molded component 300A is shown in FIG. 4. The component 300A is molded to have a generally conical base portion 306A, a generally planar blade portion 308A, a proximally facing surface 307 for attachment to the distal end of the cannula, and a proximally extending tissue plug 309.

In order to form the distal tip shown in FIG. 3, the component 300A can be hardened, such as by heat treating using an H900 heat treatment process (ASTMF899) to increase the hardness of the blade from about 28 RHC to at least about 40 RHC. After heat treating the distal tip component shown in FIG. 4, the distal tip component is mounted to the cannula 80, as shown in FIG. 5. The distal tip component 300A is joined to the cannula 80 by welding or any other suitable manner, such that the distal tip component is generally permanently attached to the cannula in the final, in-use position for use in the biopsy device.

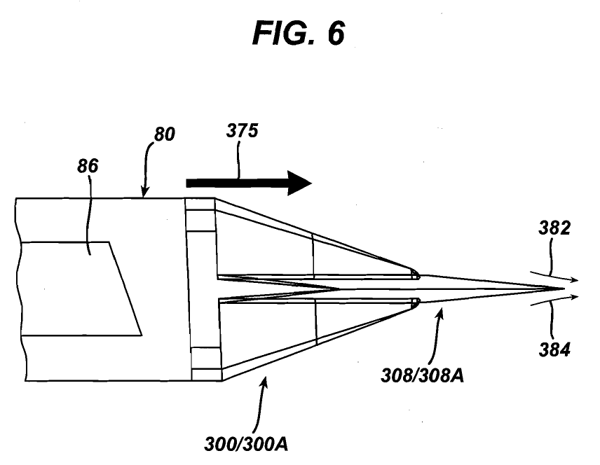

With the distal tip component 300A joined to the cannula 80, the distal tip component 300A and cannula 80 (including the internal cutter lumen of the cannula) are polished and passivated, such as by electropolishing. After electropolishing, the sharp leading edge can be formed on the blade portion 308A. The sharp leading edge can be formed as schematically illustrated in FIG. 6.

With the distal tip component 300A attached to the cannula 80, the blade portion 308A is advanced (in the direction 375 generally parallel to the longitudinal axis 81) toward a honing apparatus. The honing apparatus can remove material from blade portion 308A as indicated by arrows 382 and 384 in FIG. 6. In one embodiment, the opposite sides of blade portion 308A can be honed simultaneously, such as in the directions indicated by arrows 382 and 384, to provide facet surfaces 330A and 330B and leading edge portions 320A and 320B. The honing apparatus can be CNC honing device. Incision Tech company of Staunton, Va. provides honing/grinding processes suitable for use in forming the distal tip shown in FIG. 3.

Facet surfaces 330A can be formed on opposite sides of the blade simulatenously, and facet surfaces 330B can be formed on opposite sides of the blade simultaneously. The facet surfaces can be created by removing approximately 0.015 inch of material from the end of the blade portion 308A.

After the honing to form the facet surfaces and sharp leading edge, the cannula and distal tip 300 can be cleaned to remove burrs and grinding lubricant, such as by use of an aqueous solution and a cleaning process such as set forth in ASTM A380-99.

Embodiments described herein may be incorporated into and combined with any of the devices and components described in U.S. patent application Ser. No. 10/785,755, filed Feb. 24, 2004, entitled “Biopsy Device with Variable Speed Cutter Advance,” issued as U.S. Pat. No. 7,025,732, the disclosure of which is incorporated by reference herein, in any suitable fashion. Embodiments described herein may also be incorporated into and combined with any of the devices and components described in U.S. patent application Ser. No. 11/192,764, filed Nov. 20, 2007, entitled “Vacuum Timing Algorithm for Biopsy Device,” the disclosure of which is incorporated by reference herein, in any suitable fashion. Embodiments described herein may also be incorporated into and combined with any of the devices and components described in U.S. patent application Ser. No. 12/038,359, filed Feb. 27, 2008, entitled “Needle Tip for Biopsy Device,” the disclosure of which is incorporated by reference herein, in any suitable fashion. Suitable ways in which methods, devices, and components described herein may be combined or incorporated into the methods, devices, and components described the above-referenced patent disclosures will be apparent to those of ordinary skill in the art in view of the teachings herein.

While embodiment of the present invention have been shown and described herein, those skilled in the art will recognize that such embodiments are provided by way of example, and that numerous variations, changes, and substitutions will now occur to those skilled in the art without departing from the spirit and scope of the present invention. Further, each element disclosed may be alternatively described as a means for performing the element's function.

What is claimed:

1. A biopsy device comprising:
a cannula having longitudinal axis, a distal end, a cutter lumen, and a tissue receiving port disposed proximally of the distal end, the tissue receiving port communicating with the cutter lumen;
a tissue cutter adapted for translation within the cutter lumen to sever tissue received in the tissue receiving port of the cannula; and
a distal tip disposed at the distal end of the cannula, the distal tip comprising a blade having a hardness of at least about 40 HRC.

2. The biopsy device of claim 1 wherein the distal tip comprises a blade having a hardness of between about 43 HRC and about 45 HRC.

3. The biopsy device of claim 1 wherein the distal tip comprises a metal injection molded component.

4. The biopsy device of claim 1 wherein the blade has a sharp leading edge having a plurality of microscopic teeth.

5. The biopsy device of claim 1 wherein the blade has a leading edge, and wherein the blade has an included leading edge angle of less than about 45 degrees.

6. The biopsy device of claim 1 wherein the blade has a leading edge, and wherein the leading edge has an included leading edge angle less than or equal to about 42 degrees.

7. The biopsy device of claim 1 wherein the blade comprises a plurality of serrations, the serrations being generally aligned with the longitudinal axis of the cannula.

8. The biopsy device of claim 1 wherein the blade comprises a pair of generally oppositely facing sides, and wherein each side comprises a plurality of serrations generally aligned with the longitudinal axis of the cannula.

9. The biopsy device of claim 1 wherein the blade comprises a honed surface formed on a first side of the blade, and a honed surface on a second side of the blade, and wherein each honed surface comprises serrations extending proximally from a leading edge of the blade.

10. A biopsy device comprising:
a cannula having longitudinal axis, a distal end, a cutter lumen, and a tissue receiving port disposed proximally of the distal end, the tissue receiving port communicating with the cutter lumen;
a tissue cutter adapted for translation within the cutter lumen to sever tissue received in the tissue receiving port of the cannula; and
a distal tip disposed at the distal end of the cannula, the distal tip comprising a blade having a hardness of at least about 40 HRC, and the blade having an included leading edge angle of less than about 45 degrees.

11. A biopsy device comprising:
a hollow cannula having longitudinal axis and a distal end;
a distal tip disposed at the distal end of the cannula, the distal tip comprising a blade having a first side, a second side, and a leading edge;
wherein the leading edge is disposed in a plane extending substantially parallel to the longitudinal axis of the hollow cannula;
wherein each of the first and second sides comprises a honed surface extending from the leading edge; and
wherein each honed surface comprises a plurality of serrations, the serrations being generally aligned with the longitudinal axis of the cannula.
